(19) United States
(12) Patent Application Publication
(10) Pub. No.: US 2026/0207827 A1
(43) Pub. Date: Jul. 23, 2026

(54) A MILK CONTAINER FOR A BREAST PUMP

(71) Applicant: Willow Blossom Holdco Limited, London (GB)

(72) Inventors: Brian MCVEIGH, Bristol (GB); Christopher James IRLAM, Gloucester (GB); Jeremy William CROUCH, Malmesbury, Wiltshire (GB); Thomas Michael MALLOY, Oxford (GB); Jack Cyril BILTCLIFFE, Bristol (GB); Macdara O'SHEA, Bristol (GB); Daniel John THOMPSON, Bristol (GB)

(21) Appl. No.: 19/107,490

(22) PCT Filed: Aug. 31, 2023
(86) PCT No.: PCT/EP2023/073978

§ 371 Date: Feb. 27, 2025

(30) Foreign Application Priority Data
Aug. 31, 2022 (EP) 2212674.2

(51) Int. Cl. A61M 1/06

(52) U.S. Cl. CPC A61M 1/064; A61M 1/067; A61M 2209/088

(57) ABSTRACT

There is provided a wearable breast pump, comprising a milk pumping system for receiving milk from a user of the breast pump and a milk container comprising a flexible diaphragm. The flexible diaphragm forms part of a wall of the milk container.

### FIELD OF THE INVENTION

The present disclosure relates to a milk container for a breast pump and, in particular, to a milk container for an in-bra wearable breast pump for expressing human milk.

### BACKGROUND

A breast pump system is a mechanical or electro-mechanical device that extracts milk from the breasts of a lactating person.

Breast pumps for expressing human breastmilk are known. A vacuum is used to simulate suction generated by a feeding child. Breast pumps typically include a milk container for collecting the milk expressed from a breast.

Fully integrated wearable breast pump systems are known in the art. In such pump systems, the suction source, power supply and milk container are contained in a single, wearable device and there is no need for bulky external components or connections. Such devices can be provided with a substantially breast shaped convex profile so as to fit within a user's bra for discreet pumping, as well as pumping on-the-go without any tethers to electrical sockets or collection stations. The internal breast shield is convex to fit over a breast. Known milk containers include solid framed containers and flexible milk bags.

Many milk containers for breast pumps are prone to leakage, difficult to dry or clean, and are not discreet. In view of this, there is a need for an improved milk container to accommodate for these downfalls. Further, there is a need for a milk container which allows a base level vacuum to be generated to hold a breast pump to a user's breast, thereby providing a more efficient pumping system and reduced milk leakage.

### SUMMARY

There is provided a milk container for a breast pump as defined in the appended claims.

### BRIEF DESCRIPTION OF THE DRAWINGS

Specific embodiments are described below by way of example only and with reference to the accompanying drawings in which:

FIG. 1 shows an inward configuration milk container and breast pump according to an embodiment of the invention.

FIG. 2 shows an inward configuration milk container according to an embodiment of the invention.

FIGS. 3A-3H show an inward configuration milk container and breast pump according to an embodiment of the invention.

FIG. 4 shows an outward configuration milk container according to an embodiment of the invention.

FIG. 5A shows an over moulded outward configuration milk container according to an embodiment of the invention.

FIG. 5B shows a non-return valve and over moulded milk container configuration according to an embodiment of the invention.

FIG. 6A shows a bottom loaded inward configuration milk container according to an embodiment of the invention.

FIG. 6B shows a bottom loaded outward configuration milk container according to an embodiment of the invention.

FIG. 7 shows a bottom loaded outward configuration milk container according to an embodiment of the invention.

FIG. 8 shows a top loaded and outward configuration milk container according to an embodiment of the invention.

FIG. 9 shows a milk container and removable milk bag according to an embodiment of the invention.

FIGS. 10A and 10B show a milk container according to an embodiment of the invention.

FIG. 11 shows a venting system according to an embodiment of the invention.

FIGS. 12A and 12B show a venting system according to an embodiment of the invention.

FIG. 13 shows a venting system according to an embodiment of the invention.

FIG. 14 shows a vent valve according to an embodiment of the invention.

FIG. 15 shows a venting system according to an embodiment of the invention.

FIG. 16 shows a venting system according to an embodiment of the invention.

Embodiments and features of the present disclosure are set out in the accompanying claims.

### DETAILED DESCRIPTION OF THE EMBODIMENTS

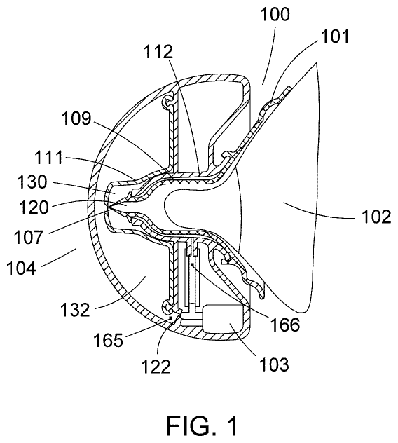

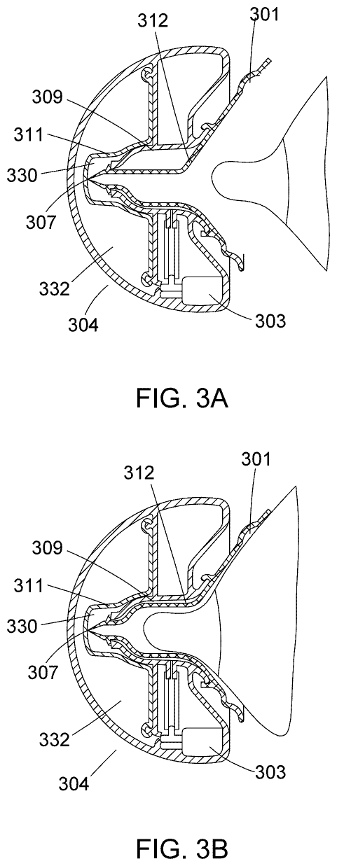

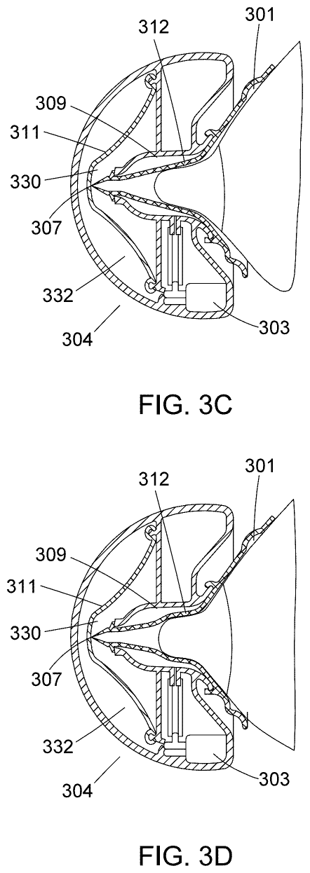

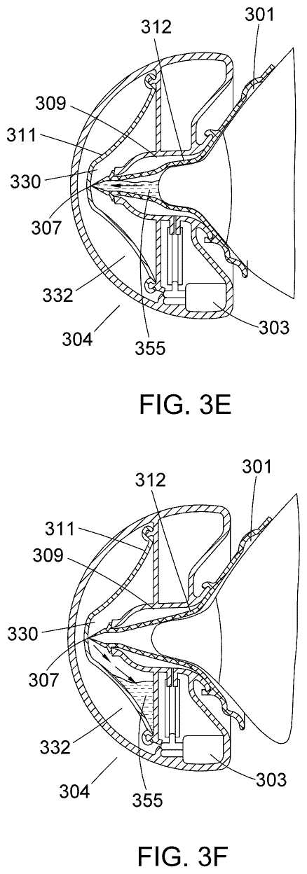

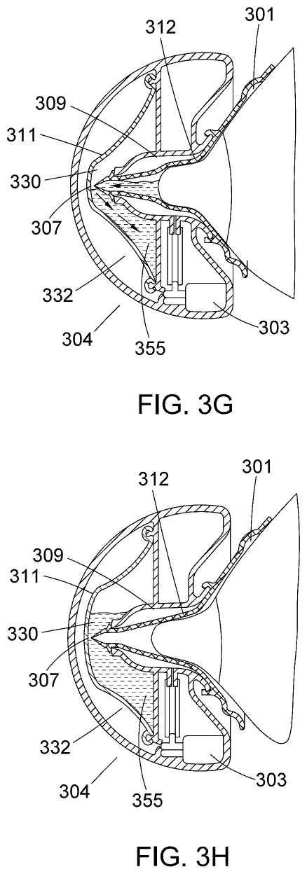

A milk container for a breast pump according to embodiments of the present disclosure is shown in FIGS. 1 to 9. Different configurations of a milk container according to different embodiments will be described with reference to FIGS. 1 to 7. Operation of the milk container with a breast pump will be described in the most detail with reference to FIGS. 3A-H, however, similar operation is achieved with each of the different embodiments, as will be understood by a person skilled in the art.

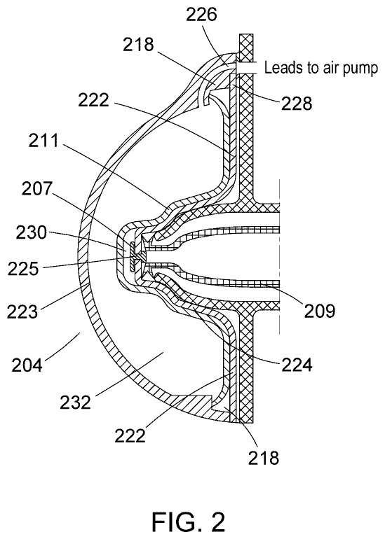

Reference will now be made to FIG. 1. Although described with reference to FIG. 1, the structures described are also relevant to FIGS. 2-9.

FIG. 1 shows a milk container 104 according to some embodiments. The milk container 104 may comprise a flexible diaphragm 111 attached to and/or located inside the milk container. The milk container 104 may be attached to a milk pumping system to receive milk from a user of the breast pump. The milk pumping system may be a breast pump 100. The breast pump 100 is a kind suitable for expressing human breast milk. The assembled breast pump 100 system may include a housing (not shown) shaped to substantially fit inside a bra. The housing is designed to enclose all the components of the breast pump 100, optionally including the milk container 104. The housing may be shaped to discreetly fit underneath a user's clothing or to be worn inside a bra. The housing may comprise a breast shield 101 for fitting to a user's breast 102, at least one air-pump 103 for generating a vacuum. The housing may also includes a rechargeable battery and control electronics (not shown). The milk container 104 may be connected to the housing. The milk container 104 may be connected to the housing such that it provides a surface shaped to continue a curved or breast-like shape of the pump. The milk container 104 may be a re-useable milk container. The milk container is therefore for an in-bra wearable breast pump.

In some embodiments, as shown in FIGS. 1 to 8, the milk container 104 is a rigid bottle or container. The milk container 104 is designed to receive the breast milk from a user via a nipple tunnel 109 and store the breast milk whilst the user continues to operate the breast pump 100. The milk container 104 can be a re-useable milk container that is connected to the housing.

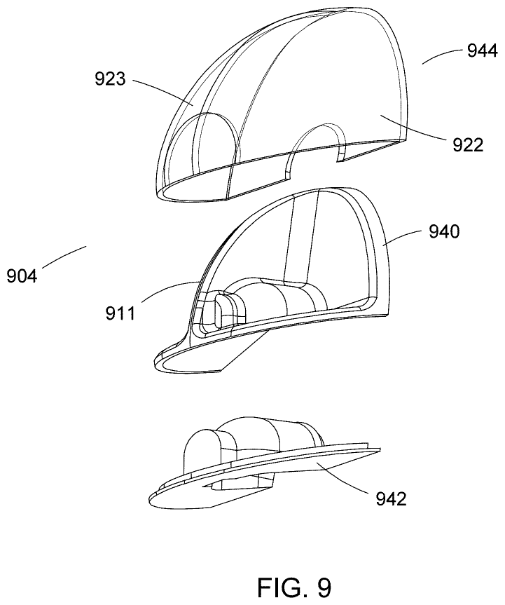

In some embodiments, the milk container 104 may be a removable milk bag. In this configuration the milk bag may be single-use or multi-use. The milk bag may be configured to fit within a milk bag housing to support the milk bag when collecting milk. A removable milk bag is described in more detail in relation to FIG. 9.

The milk container 104 may be configured to be attached to a breast pumping chamber. The breast pumping chamber is part of the breast pumping system 100. The part of the breast pumping system 100 which is the breast pumping chamber may be considered to be a nipple tunnel 109 and a non-return valve 107 (as shown in FIG. 1). The nipple tunnel 109 is shaped to receive a nipple. For example, the nipple tunnel 109 can be generally cylindrically shaped in its full volume. The milk container 104 may comprise a first opening 120 for receiving milk from the breast pump. The milk container 104 receives milk at the first opening 120 along the milk path. In some embodiments, milk only flows along the milk path through the breast shield 101 and the nipple tunnel 109, and then directly into the milk container 104. The distance required for milk to flow through the breast shield 101 and the nipple tunnel 109 can be minimized. For example, the length of the nipple tunnel 109 taken from the breast shield 101 can be minimized. The first opening 120 for receiving milk from the breast pump may be located anywhere in the wet section 130 of the milk container.

A milk path is hereby defined as the route that milk flows from the nipple through a breast shield and into the milk container 104. The milk container 104 comprises a wet section 130 and a dry section 132. The milk is comprised only in the wet section 130 of the milk container 104 and not the dry section 132. Therefore, the milk path flows from the breast shield to the wet side of the milk container.

In some embodiments, to create a negative pressure in the milk container 104, and to draw milk along the milk path to the container, an air pump 103 is used to pump air from the milk container. The air pump 103 removes air from the dry side of the milk container. The air pump 103 is connected to the dry side of the milk container via an air path. The air path is the route that air takes from the milk container 104 to the air-pump 103 or vice versa, the air path will only involve the dry section 132 of the milk container and not the wet section 130. The milk container 104 provides a hermetic seal to both the air pump 103 and the breast shield so that a vacuum can be drawn inside the bottle without any air leaks. Similarly, the hermetic seal prevents any milk from leaking out of the bottle. A hermetic seal is any type of sealing that makes a given object airtight (preventing the passage of air, oxygen, or other gases). A hermetic seal is also fluid-tight, thereby preventing the passage of milk or other fluids.

The milk container 104 may be configured as being in-bra wearable. The breast pump 100 may also be a self-contained, in-bra wearable device.

In some embodiments, the only parts of the system that come into contact with milk in normal use are the breast shield 101 of the breast pump 100 and the milk container 104 and any portion of the breast pump 100 which connects the breast shield 101 and the milk container 104, such as a nipple tunnel 109 and non-return valve 107. In some embodiments, milk only flows along a milk path through the breast shield 101 and then directly into the milk container 104. Milk does not contact any part of the housing, for maximum hygiene and ease of cleaning. The configuration of the milk container 104 allows for easy assembly, disassembly and replacement of the milk container. This also allows for the milk container 104 to be easily washed and/or sterilised. The milk container may therefore be efficiently and easily cleaned and re-used.

In some embodiments, a non-return valve 107 is provided at the downstream end of the nipple tunnel 109. The non-return valve 107 is designed to allow fluid to pass in only one direction. Therefore, in some embodiments breast milk is allowed to pass from the nipple tunnel 109 of the breast shield 101 to the milk container 104 where it is stored. The air pump 103 draws milk or air from the nipple tunnel 109 of the breast shield 101 to the milk container 104 and it is allowed to flow past the non-return valve 107. The valve is shaped so that when fluid (i.e. milk or air) enters the valve its pressure holds the closing mechanism open. However, milk or air flow from the milk container 104 to the nipple tunnel 109 of the breast shield 101 is blocked due to pressure of the milk or air on the non-return valve 107. The valve is designed so that if the fluid attempts to flow back through the non-return valve 107 in the wrong direction, the closing member is forced back over the entrance of the non-return valve 107 preventing any flow. This ensures that when base level vacuum is not applied, no fluid leaks from the milk container 104 back into the nipple tunnel 109 and towards the user.

The non-return valve 107 may be located either at or intermediate to the exit of the nipple tunnel 109 and/or the entrance to the milk container 104. In some embodiments, the non-return valve 107 is located at the entrance of the milk container 104 to avoid any milk leaking out of the milk container 104 and into the tube. The breast pump 100 may also comprise at least one support means or tube 106 arranged internally to receive and hold the non-return valve 107.

In some embodiments, the non-return valve 107 may be an umbrella valve. Umbrella valves are elastomeric valve components that have a diaphragm shaped sealing disk (i.e. an umbrella shape). When mounted in position, such as in the tube, the convex diaphragm flattens out against the valve seat and absorbs a certain amount of seat irregularities and creates a sealing force. The main advantage of an umbrella valve is that they can be preloaded with a closing force so when the milk container 104 is removed away from the vacuum source (for example, at the end of the pumping session), it remains shut under atmospheric pressure. This provides the advantage of preventing milk leakage when transporting and storing collected milk.

In some embodiments, a duck bill valve may be used as a non-return valve 107. Duckbill valves are one-piece, elastomeric components that act as backflow prevention devices or one-way valves. They have elastomeric lips in the shape of a duckbill which prevent backflow and allow forward flow. The main advantage of duckbill valves over other types of one-way valves is that duckbill valves are self-contained i.e. the critical sealing function is an integral part of the one-piece elastomeric component as opposed to valves where a sealing element has to engage with a smooth seat surface to form a seal. When a duck bill valve is used as a non-return valve 107, the duck bill valve will generally be at least partially open under atmospheric pressure, making leaks more likely when a vacuum is not applied to the system or during transportation or storage of the milk container 104.

In some embodiments, a flap valve may be used as a non-return valve 107. A flap valve ensures that fluid can pass through the valve in one direction only as the pressure of the fluid pushes the swinging door open. When the pressure drops below a certain point, the flap closes.

The air pump 103 may be a mechanical air pump 103 designed to either extract air from a breast pump 100 system or insert air into a breast pump 100 system. In some embodiments, the air pump 103 is configured to draw air out of the breast pump 100 system and create a negative air pressure in the nipple tunnel 109.

A negative air pressure differential is defined as any pressure below that of the surrounding air environment. In other words, a negative air pressure differential is a pressure lower than the system of the breast pump 100 when the air pump 103 is not in use. A typical air pressure, or atmospheric pressure, of a standard environment is 760 mmHg, therefore a negative air pressure may be defined as any pressure lower than 760 mmHg.

In some embodiments, the air pump 103 may also be configured to generate a positive air pressure in the nipple tunnel 109. For example, a positive air pressure may be used to assist with emptying or evacuating elements of the breast pump 100. In particular, a positive air pressure can be used to expel milk from the milk container 104.

The air pump 103 may be a rotary diaphragm pump. A rotary diaphragm pump is a positive displacement pump that uses a combination of the reciprocating action of a flexible diaphragm (e.g. made from silicone, rubber, thermoplastic or Teflon) and suitable valves on either side of the diaphragm to pump a fluid. In some embodiments, the rotary diaphragm pump pumps air. A rotary air pump provides a cost effective and easy way to reach desired pressures. In this case, the air pumping subsystem may either be configured as an open loop or closed loop pumping subsystem. The rotary diaphragm pump used may be a standard rotary diaphragm pump as is well known for use in breast pump 100.

Alternatively, the air pump 103 may be a piezoelectric pump. Piezoelectric air pumps (or piezo pumps), operate silently (e.g. outside the range of human hearing) and with minimal vibrations. Due to their low noise, strength and compact size, piezoelectric pumps are ideally suited to the embodiment of a small, wearable breast pump. However, piezo pumps generate higher heat as compared to, for example, rotary diaphragm pumps. Reducing the air volume in the system improves the efficiency of the pump, and the pump therefore generates less heat. When a piezoelectric pump is used, the air pumping subsystem may also either be configured as an open loop or closed loop pumping subsystem.

Other possible types of pumps are also feasibly usable in some embodiments. For example, a peristaltic or vein pump could also be used.

In some embodiments, the pump is housed within the in-bra breast pump 100. However, the pump may also be housed separately and connected to the in-bar architecture by simple tubing.

The wearable breast pump 100 may be configured to operate quietly in normal use. A cavity containing the air pumping subsystem (including the air pump 103, solenoid valve 105), may be sealed and comprise other noise reduction technology so as to further attenuate sound.

The milk container 104 may comprise a second opening 122 for connection to the air pump 103. The second opening 122 forms part of the air path for air to move from the milk container 104 to the air pump 103 or vice versa. The first opening 120 may also be configured to allow easy pouring of collected milk out of the device, for example via a pouring spout. In such cases the first opening 120 may also be stopped to allow for storage of the milk.

In some embodiments, the milk container may comprise a separate pouring opening for pouring collected milk out of the milk container. This pouring opening may be a third opening. In such embodiments, the first opening is not used for pouring milk. The pouring opening may be configured to allow easy pouring of collected milk out of the device, for example via a pouring spout. It may also be stopped to allow for storage of the milk.

As explained above, milk is drawn into the wet side of the milk container. In some embodiments, the flexible diaphragm 111 acts as a milk barrier for preventing milk from exiting the milk container. For example, the flexible diaphragm prevents milk from exiting the milk container and reaching the air-pump 103 offering back-flow protection for the breast pump 100. The flexible diaphragm 111 divides the container into a wet section 130 and a dry section 132. The wet section 130 is suitable for receiving breast milk. The dry section 132 is suitable for holding air. The flexible diaphragm 111 is designed such that there is a hermetic seal between the wet section 130 and the dry section 132, such that milk collected in the wet section 130 cannot pass through into the dry section 132.

In order to separate the wet section 130 for receiving breast milk and the dry section 132 for holding air, the flexible diaphragm 111 forms a hermetic seal around an internal wall of the milk container. An internal wall of the milk container 104 may be located at any position in the milk container. The flexible diaphragm 111 may be configured such that it passes circumferentially around the milk container 104 (as shown in FIG. 1). The hermetic seal may prevent milk reaching the dry section 132 of the milk container 104 and prevent air from escaping from the dry section 132 to the wet section 130 of the milk container.

The flexible diaphragm 111 may intersect the milk container. That is, the flexible diaphragm 111 may divide the milk container into two sections. In other words, the flexible diaphragm 111 may divide the internal volume of the milk container into two sections. The two sections need not be equal in size. In some examples, however, the flexible diaphragm 111 may bisect the milk container. That is, the flexible diaphragm 111 may divide the milk container into two substantially equal sections. Due to the flexible nature of the diaphragm and the operation of the breast pump, the two sections may change in volume during use. A first section is the wet side of the milk container and the second section is the dry side.

The wet side of the milk container may comprise the flexible diaphragm and the internal side of an external wall of the milk container. The wall of the milk container may be a rigid wall. In FIGS. 1 to 8, the wet side is defined by the diaphragm and a portion of the wall of the milk container.

In some embodiments, the flexible diaphragm may attach along its perimeter to the external wall (i.e. the external surface) of the milk container 104. This is to aid in forming a hermetic seal around the internal wall in the milk container. In such examples, the flexible diaphragm remains internal to the milk container but the attachment and seal form on the external wall of the milk container.

The flexible diaphragm 111 may also take the form of a flexible pouch or milk bag which also prevents milk reaching the dry section 132 of the milk container 104 and prevents air from escaping from the dry section 132 to the wet section 130 of the milk container.

The flexible diaphragm 111 may be made from a flexible material and is designed to deform when a pressure change is caused by the air pump 103. That is, the flexible diaphragm 111 is designed to deform when acted upon by a vacuum. For example, when the air pump 103 is turned on, it acts upon the flexible diaphragm 111 to deform the material. Pumping air via the air pump 103 from the dry side causes the flexible diaphragm 111 to move into the dry side, transferring negative pressure to the wet side and along the milk path. The flexible diaphragm 111 therefore creates suction along the milk path, causing milk to be expressed.

The flexible diaphragm 111 may be made entirely from silicone or may be made in part by silicone. As will be appreciated, any suitable flexible material or combination of materials may also be used, such as a thermoplastic elastomer, a thermoplastic polyurethane, polyester, a flexible or an elastic elastomer such as natural or synthetic rubber. In some embodiments, the flexible diaphragm is made from silicone, such that it may be easily dried and cleaned by a user of the breast pump 100.

The flexible diaphragm 111 may be completely or partially removable from the milk container 104. This is to allow for ease of cleaning. When the milk container is in use, the flexible diaphragm 111 may be secured along its perimeter to form the hermetic seal. As will be appreciated, the flexible diaphragm 111 may be secured along its perimeter in a number of different ways. In one example, the flexible diaphragm 111 may be secured along its perimeter between two attachments, the flexible diaphragm trapped in the middle of the two attachments. The two attachments may optionally clip together by a mechanical clip. Alternatively, the two parts may hold the flexible diaphragm 111 in place by the friction of the flexible diaphragm alone. In another example, the two attachments may screw together with a screw attachment.

In the embodiments where the flexible diaphragm 111 is completely removable from the milk container, the diaphragm can completely detach from the milk container. In the embodiments where the flexible diaphragm is partially removable from the milk container, the diaphragm can detach along some of its perimeter whilst being permanently attached along the remaining perimeter. For example, the diaphragm may be able to detach along half the perimeter whilst being permanently attached along the remaining half of its perimeter.

The flexible diaphragm is activated by an air pump applying negative pressure on the dry side of the diaphragm. In some examples, in a relaxed state the diaphragm has a non-flat profile, such as a curved or undulating profile. This enables the diaphragm to move without requiring the diaphragm material to stretch.

In some embodiments, the geometry of the flexible diaphragm 111 may be configured such that the flexible diaphragm 111 has a very low spring force. Additionally or alternatively, the material of the flexible diaphragm 111 may also be chosen such that the flexible diaphragm 111 has a very low spring force. A very low spring force of the diaphragm may be where the spring force is less than the weight of the volume of milk that can fit in the container, in any position of the diaphragm. As will be appreciated, the flexible diaphragm 111 with a very low spring force acts in a neutral way. A flexible diaphragm 111 with a very low spring force prevents milk squirt/ejection when collected milk is poured out of the device. A flexible diaphragm 111 with a very low spring force also improves the efficiency of the pumping system, as compared to a flexible diaphragm 111 with a high spring force.

In some embodiments, the geometry or material of the flexible diaphragm 111 may be configured to have the flexible diaphragm of a spring force which delivers a high flow pour when the milk is poured out of the milk container. A high spring force of the diaphragm may be where the spring force is greater than the weight of the volume of milk that can fit in the container, in any position of the diaphragm. This configuration reduces the time taken to decant milk out of the milk container.

Since in some embodiments the diaphragm 111 is attached to the interior perimeter of the container, the diaphragm will have a profile similar to the profile of the container. That is, the diaphragm may have a hemispherical or half-ellipsoid profile to be attached within a breast shaped hemispherical or half-ellipsoid shaped milk container.

In some embodiments, the flexible diaphragm 111 is located between the first 120 and second opening 122 and forms a seal (i.e. a hermetic seal). The seal may be considered to be between the first 120 and second 122 openings and separates the wet section 130, the milk path, from the dry section 132, the air path. The seal is a hermetic seal and may extend along the perimeter of the inner wall of the milk container 104. As explained above, the flexible diaphragm 111 is configured to deform under a negative pressure from the air pump 103 and create a suction along the milk path into the milk container 104.

The breast pump 100 may also comprise a second flexible diaphragm 112. The breast shield 101 of the breast pump 100 may comprise the second flexible diaphragm 112. The second flexible diaphragm 112 is also made from a flexible material and is designed to deform when a negative air pressure is drawn by the air pump 103. It should be noted that only part of the breast shield 101 forms the second flexible diaphragm 112, the second flexible diaphragm 112 is a sealed deformable section of the breast shield which is not affixed in place.

The flexible diaphragm 111 may be referred to as the first flexible diaphragm or simply the flexible diaphragm. Alternatively, the second flexible diaphragm 112 may be seated against a diaphragm holder that is in close communication with the nipple tunnel 109, the diaphragm deforming in response to changes in air pressure caused by the air pump to create negative air pressure in the nipple tunnel. The second flexible diaphragm 112 may be substantially circular and is configured to self-seal under the negative air pressure to a substantially circular diaphragm holder that is part of the housing or a recess in the rear surface of the housing.

When the air pump 103 is activated, negative air pressure differential is created between the air pump 103, the two diaphragms 112 and 111, such as those in the milk container 104 and/or the breast shield 101, thereby applying negative pressure differential to the nipple, drawing milk from the breast, and collecting it inside the milk container 104. The negative air pressure differential has the meaning described above.

The air pump 103 may be configured to pump at a wide range of different levels of intensity. A first level of pumping is provided to generate the base level vacuum inside the nipple tunnel 109. A second level of pumping is provided to generate a pumping vacuum to stimulate the breast tissue and initiate milk expression from the breast. The second level of pumping is more intense than the first level. This is because a greater negative air pressure must be generated for when the air pump 103 is expressing milk from the user's breast 102, compared to when only the base level vacuum desired pressure is required.

The first level of pumping is configured to produce a negative air pressure from −15 to −70 mmHg (relative to atmospheric pressure) inside the nipple tunnel 109. In one embodiment, the first level of pumping is configured to produce a negative air pressure from −30 to −60 mmHg (relative to atmospheric pressure) inside the nipple tunnel 109. In another embodiment, the first level of pumping is configured to produce a negative air pressure of −50 mmHg (relative to atmospheric pressure) inside the nipple tunnel 109. The above pumping pressures are disclosed as merely examples, and the skilled person would understand that other feasible ranges are possible. The first level of pumping may also be determined by a user's preferences and input via a connected device to the breast pump.

The second level of pumping is configured to produce a negative air pressure from −150 to −300 mmHg—(relative to atmospheric pressure) inside the nipple tunnel 109. In one embodiment, the second level of pumping is configured to produce a negative air pressure from −200 to −280 mmHg (relative to atmospheric pressure) inside the nipple tunnel 109. In another embodiment, the second level of pumping is configured to produce a negative air pressure from −230 to −280 mmHg (relative to atmospheric pressure) inside the nipple tunnel 109. The above pumping pressures are disclosed as merely examples, and the skilled person would understand that other feasible ranges are possible. The first level of pumping may also be determined by a user's preferences and input via a connected device to the breast pump.

In some embodiments, pressure sensor/s may be provided in the system. A first pressure sensor 165 may be provided between the air pump 103 and the milk container 104. A second pressure sensor 166 may be provided between the air pump 103 and the breast shield 101.

The pressure sensors can be used to actively monitor the pressure at both the first and second diaphragms.

The pressure sensor may be used to monitor the level of the base level vacuum and assist in the measurement of milk collection in the milk container 104, by calculating pressure changes in the milk container 104.

Reference will now be made to FIG. 2. Although described with reference to FIG. 2, the shape of the external surfaces of the milk container presently described is also applicable to FIGS. 1-7.

FIG. 2 shows a milk container 204 according to some embodiments. The external surfaces of the milk container 204 may be shaped as depicted in FIG. 2, this external structure is similar to the external structure shown in FIGS. 1 and 3-9. The milk container may be a breast shaped hemispherical or half-ellipsoid shape, comprising a substantially flat section 222 and a domed section 223, wherein the flat section and domed section meet at an edge 228. The shape of the container may be described as egg shaped or pebble shaped.

The domed section 223 of the milk container may be shaped to mimic a breast-shape and facilitate the milk container 204 to be in-bra wearable and offers the user a discrete profile when wearing the breast pump 100.

The substantially flat section 222 may optionally comprise an internal protrusion 224 in a centred position with respect to the flat section. The internal protrusion 224 is away from the edge 228 where the flat section 222 meets the domed section 223. The internal protrusion 224 protrudes within the volume of the milk container, creating an indented portion of the substantially flat section 222. The internal protrusion 224 may be of different sizes depending on the configuration of the milk container and to ensure that it matches the geometry of the nipple tunnel 109.

Now reference will be made to FIG. 2 to describe the example configuration of the milk container 204 depicted in FIG. 2. The described milk container and diaphragm configuration is applicable to FIGS. 1 and 3A-H.

Similarly to as described in reference to FIG. 1, the milk container 204 may comprise a wet side and a dry side. A milk path is defined as the route that milk flows from the nipple through a breast shield and into the milk container 204. The milk is contained only in the wet section 230 of the milk container 204 and not the dry section 232. The air path is the route that air takes from the milk container to the air-pump or vice versa, the air path will only involve the dry section 232 of the milk container and not the wet section 230.

In some embodiments, when the milk container 204 is positioned such that it is configured to receive milk from the user of the breast pump, the milk container is configured such that the dry section 232 is located on the side away from the user and the wet section 230 is located on the side towards the user. In other words, the dry section 232 is outwardly facing and the wet section 230 is inwardly facing. This means that the milk filling the milk container 204 when the breast pump is in use is less visible to the user, since the milk is collecting on the side towards the user and the dry section 232 substantially surrounds the wet section 230. However, having an inward facing configuration for the wet section of the milk container 204 is beneficial from a mechanical perspective, since this corresponds to the side from which milk is delivered to the milk container 204 from the breast via the milk path. This is the configuration also depicted in FIGS. 1 and 3A-H.

In some embodiments, the milk container 204 is formed of a first housing piece and a second housing piece, wherein the first housing piece and second housing piece meet at an edge 228. In this configuration, the first housing piece 222 forms the substantially flat section 222. The second housing piece 223 forms the domed section 223 of the milk container. In other words, the first housing piece is shaped to be substantially flat and the second housing piece is shaped as a substantially breast shaped hemispherical or ellipsoid dome.

The first housing piece may comprise an attachment portion 218 to receive a portion of the flexible diaphragm 211 and seal the flexible diaphragm to the first housing piece. The attachment portion 218 is located along at least a portion of the edge 228 and hermetically seals the flexible diaphragm to the edge 228. The attachment portion may extend the entire circumference of the edge 228 between the first housing piece and the second housing piece or it may extend over a portion of the edge 228 between the first housing piece and the second housing piece.

The first housing piece may comprise an internal protrusion 224, the shape of the internal protrusion is configured such that the milk container 204 can attach to the breast pumping chamber. The first housing piece remains substantially flat but with the internal protrusion 224. The internal protrusion 224 is located along an axis extending from the substantially center of the domed section 223.

The milk container 204 may comprise a first opening 225 configured to receive milk from a user's breast and expel the milk into the wet section 230. The first opening 225 is located along an axis extending from the substantially center of the domed section 223. The first opening 225 may be located at the center of the internal protrusion 224 to maximise the compact nature of the breast pump 100. The first opening 225 allows milk to flow via the milk path from the nipple and the nipple tunnel 209 from outside the milk container to within the milk container 204. The first opening 225 may be a small circular hole with a radius of the order of millimetres, for example 2 mm-4 mm. The first opening 225 may also be configured to allow easy pouring of collected milk out of the device. In such cases the first opening 225 may also be stopped to allow for storage of the milk.

The milk container 204 may also comprise a second opening 226 configured to provide an air path to an air pump for generating a vacuum. The second opening 226 may be located at the edge of the first housing piece and second housing piece. As will be appreciated, second opening 226 may be located anywhere in the dry section of the milk container.

The second opening 226 allows the generation of the vacuum within the milk container, the air pump is outside of the milk container 204. When the milk container 204 is positioned such that it is configured to receive milk from the user of the breast pump, the second opening 226 may be located at the top of the milk container, at the bottom of the milk container or at any point around the circumference of the edge between the first and second milk container housings. As will be appreciated, the second opening 226 may be located anywhere in the dry section 232. The second opening 226 may be a small circular hole with a radius of the order of millimetres, for example 1 mm-2 mm.

The first 225 and second 226 openings may be adapted to fit onto conduits (i.e. tubes) for the delivery of milk and air respectively. The first 225 and second 226 openings may also comprise sealing interfaces. For example, energising lip seals or compression seals.

The flexible diaphragm 211 may be integrally formed meaning it is formed from a single piece of material. The flexible diaphragm 211 may be transparent or optically clear to allow the user to see milk is being expressed and collected. In other examples the flexible diaphragm 211 may be opaque. In some examples, the flexible diagram may be entirely flexible. In other examples, the flexible diagram may comprise inflexible elements to aid attachment to the milk container.

This configuration of the milk container is shown in FIG. 3H. In FIG. 3H, the milk 355 is kept on the wet section 330 of the milk container, the wet section of the milk container located on the side towards the user. The wet section of the milk container is towards the breast and nipple.

Reference will now be made to FIGS. 3A-H.

FIGS. 3A-H show the wearable breast pump system including a first flexible diaphragm 311 and a second flexible diaphragm 312. FIGS. 3A-3H represent the process of extracting milk from a breast by use of a milk container and breast pump. Each of FIGS. 3A-H represent succeeding points in time, starting with FIG. 3A and ending with FIG. 3H.

FIGS. 3A-H depict a configuration of milk container similar to the milk container depicted in FIGS. 1 and 2. As already discussed, the milk container 304 comprises a wet section 330 for receiving breast milk and a dry section 332 for holding air and the flexible diaphragm 311 separates the wet section and the dry section. In the example of FIGS. 3A-H, when the milk container is positioned such that it is configured to receive milk from the user of the breast pump, the dry section 332 is located on the side away from the user and the wet section 330 is located on the side towards the user. This configuration of the milk container is shown clearly in FIG. 3H. In FIG. 3H, the milk 355 is kept on the wet section 330 of the milk container 304, the wet section of the milk container located on the side towards the user. The wet section 330 of the milk container 304 is towards the breast and nipple.

A breast positioned away from the breast shield is depicted in FIG. 3A, the nipple outside of the nipple tunnel 309. A flexible diaphragm 311 is provided inside the milk container to hermetically seal a dry section 332 from a wet section 330. The initial set up of the breast pump system takes place. The initial set up of the breast pump system may include calibration of the milk container 304, calibration and resetting of any pressure sensors in the system and a start-up cycle to set up the air pump 303. Haptics and visual indicators may be used to confirm that the breast pump is properly assembled and ready to start stimulation mode.

A breast positioned against the breast shield is depicted in FIG. 3B, the nipple placed inside the nipple tunnel 309. When a nipple is placed inside the nipple tunnel 309, as in FIG. 3B, the pump 303 is actuated. The user initiates the pumping process by applying the breast shield 301 and fitting the breast shield 301 comfortably to the surface of the breast. The breast shield 301 can be applied by inserting the nipple into the entrance of the nipple tunnel 309. Then a low level pump vacuum may be applied (not shown in FIGS. 1 to 7) to achieve a base level vacuum in the nipple tunnel 309. The flexible diaphragm 311 is pulled away from its rest position and into the milk container 304 (see FIG. 3C).

In an embodiment where the nipple tunnel 309 is flexible and doubles as the second diaphragm 312, the user can apply pump vacuum to open the nipple tunnel 309 and then subsequently place their nipple into the opening and then relax the flexible nipple tunnel 309 before drawing a base level vacuum to close, and ultimately seal the flexible breast shield 301 to the user's nipple. The base level vacuum and pulling force from the flexible diaphragm 311 removes any excess air from the nipple tunnel 109 and causes the breast shield 312 (i.e. the second flexible diaphragm) to contract around the breast. The base level vacuum is defined by a pressure lower than the atmospheric pressure and the pressure change caused by a base level vacuum (i.e. negative base level vacuum pressure) causes breast shield 301 to contract around the breast.

The pump actuating causes the second flexible diaphragm 312 to expand or contract radially, this is depicted in FIG. 3C. The second flexible diaphragm 312 expanding or contracting radially in turn causes the nipple to be stimulated and/or to be drawn into the nipple tunnel 309, this is depicted in FIG. 3D. When the nipple is stimulated, the breast tissue is stimulated, and this causes the expression of breast milk and breast milk is expressed into the nipple tunnel, this is depicted in FIG. 3E. Once the pumping pressure is applied, the breast tissue is stimulated, and the user gradually begins expressing milk 355 into the nipple tunnel 309, as shown in FIG. 3E. During this step additional biomimetic pumping sequences may be applied to the flexible diaphragm 311 to mimic the motion of a baby's mouth during breast feeding. This may trigger the expression of milk 355 or enhance the volume of milk produced. FIGS. 3E-3G depict arrows to show the milk path during expression.

Next, as shown in FIG. 3F milk 355 passes from the nipple tunnel 309 into the wet section 330 of the milk container 304. This is achieved, for example, by applying a pressure differential to the first diaphragm to generate a vacuum in the wet section of the milk container 104. Alternatively, if a base level vacuum is applied, milk 355 will pass into the wet section 330 of the milk container 304 causing the pressure to normalise and the base level vacuum will need to be topped up. Further, alternatively, milk 355 will pass into the wet section 330 of the milk container 304 caused by the opening of a bleed valve to allow the milk 355 to flow from the nipple tunnel 309 into the milk container 304. The milk 355 passes along the milk path and through the non-return valve 307 and ends up in the wet section 330 of the milk container.

These steps shown in FIG. 3C to 3F repeat until the milk container 304 is full of milk or the pumping session otherwise ends. FIGS. 3G and 3H show the milk container 304 becoming increasingly full of milk 355. Inserting milk into the wet section 330, thereby causes the area of the dry section 332 to decrease and the area of the wet section to increase. The flexible diaphragm is configured to move and deform as milk 355 is expressed into the wet section 330 of the milk container to accommodate the milk. The flexible diaphragm 311 progressively moves outwards towards the front face of the milk container 304 to accommodate the expressed milk 355.

Whilst this process is described in relation to a milk container with a wet section for receiving breast milk with an inward configuration, the skilled person would understand that the same process steps are followed for an outward configuration.

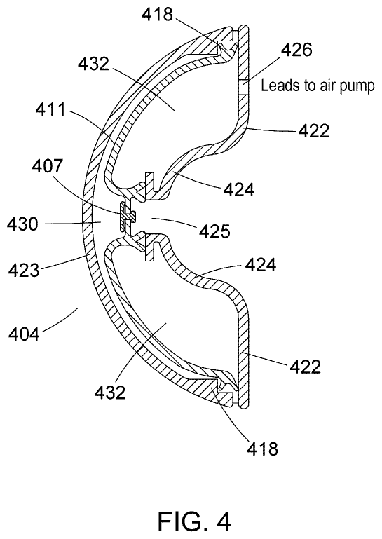

Reference will now be made to FIG. 4.

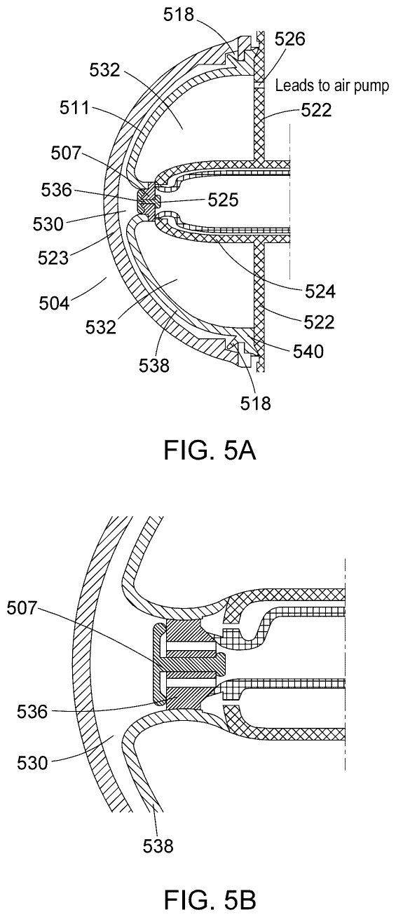

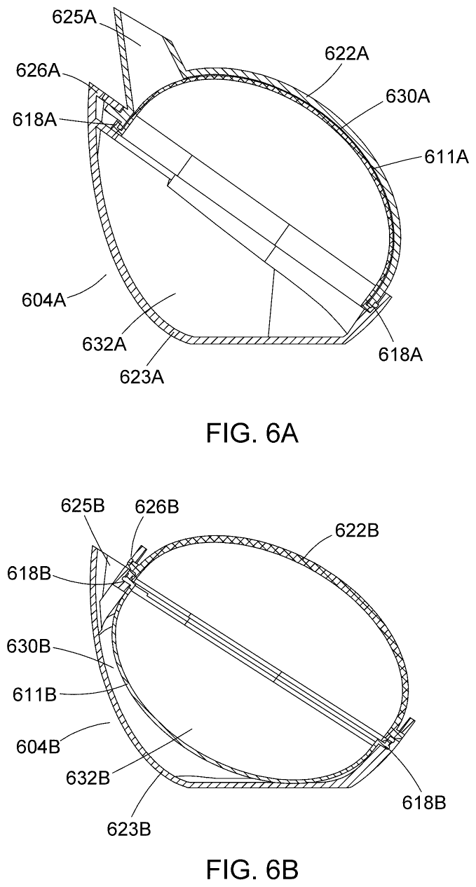

FIG. 4 shows an example configuration of a milk container 404 according to some embodiments. When the milk container 404 is positioned such that it is configured to receive milk from the user of the breast pump, the wet section 430 is located on the side away from the user and the dry section 432 is located on the side towards the user. This configuration is the same as the examples shown in FIGS. 5A-B and FIG. 6. This configuration is an outward configuration. This configuration is different to the examples of FIGS. 1-3H where the milk container is configured such that the dry section is located on the side away from the user and the wet section is located on the side towards the user (inward configuration). This offers the benefit to the user that they can easily see the milk collecting in the milk container 104 while pumping as the wet section 430 is exposed and not surrounded by the dry section 432.

In the example of FIG. 4, the wet section 432 is configured to be visible to the user when the milk container 404 is in use, that is when it is positioned to receive milk from the user of the breast pump.

The flexible diaphragm 411 is configured to move and deform as milk is expressed into the wet section 430 of the milk container to accommodate the milk. The flexible diaphragm 411 progressively moves inwards towards the backward face of the milk container 402 to accommodate the expressed milk (i.e. towards the user's chest when the breast pump is in use).

The wet section 430 of the milk container 404 may form a ring-shaped chamber. That is, the chamber extends the entire circumference of the milk container 404.

In a similar manner to previously described in relation to FIG. 2, the milk container 404 shown in FIG. 4 may comprises: a first opening 425, a second opening 426, an attachment portion 418, a domed section 423, and a substantially flat section 422 which comprises an internal protrusion 424.

With reference to FIGS. 5A and 5B, the flexible diaphragm 511 may comprise a rigid outer ring, a flexible diaphragm portion 538 and a rigid central core 536. In this example configuration, these components of the flexible diaphragm 511 may be over-moulded together. As will be appreciated, other types of moulding may be used, for example two-shot injection moulding.

The rigid central core 536 may comprise a non-return valve 507. The rigid outer ring 540 extends circumferentially around the domed section 523. The rigid outer ring 540 is located at the edge of the milk container 504. The rigid central core 536 is located along an axis extending from substantially the center of the domed section 523. The flexible diaphragm portion 538 connects the rigid outer ring 540 and the rigid central core 536. The milk path passes through the rigid central core 536 to deliver milk from a user's breast into the wet section 530. That is, the milk path is through the rigid central core 536.

With reference to the example of FIG. 5A, as the milk flows into the wet section 530 of the milk container 504 and the milk fills up the milk container, the flexible diaphragm portion 538 of the flexible diaphragm 511 will flex and move towards the user. The rigid central core 536 is fixed and rigid, it does not move throughout the milking process.

FIG. 5B depicts a close view of the over-moulded non-return valve 507, rigid central core 536, and flexible diaphragm portion 538.

In some embodiments, the flexible diaphragm 511 comprising a rigid outer ring, a flexible diaphragm portion 538, and a rigid central core 536 over-moulded together may also be present in other configurations of milk container. For example, this feature may be present in the milk container of any of FIGS. 1-4 and 6.

The flexible diaphragm 511 may comprise a rigid outer ring, a flexible diaphragm portion 538 and a rigid central core 536 over-moulded together. The over-moulding of these parts aids sealing and usability of the milk container. Over-moulding aids in usability in that it reduces the number of assembly steps required by the user and makes those steps quicker and easier. By combining the diaphragm with a rigid part, the user is not responsible for ensuring these components are assembled correctly and that the seal between them has been made. It can also add stiffness to an otherwise flexible part, making it easier to handle.

In a similar manner to previously described in relation to FIG. 2, the milk container 504 shown in FIG. 5A may comprise: a first opening 525, a second opening 526, an attachment portion 518, a domed section 523, and a substantially flat section 522 which comprises an internal protrusion 524.

As shown in FIG. 4, the milk container 404 may be positioned such that when it is configured to receive milk from the user of the breast pump, the milk container spans at least a portion of a front section of the breast pump. The front section is outwardly facing away from the user when the milk container is positioned such that it is configured to receive milk from the user of the breast pump.

The front loaded configuration may be applied to a milk container with either an inward or outward facing wet section of the milk container.

With reference to FIGS. 6A and 6B, the milk container is positioned such that it is configured to receive milk from the user of the breast pump, and the milk container spans at least a portion of a bottom section of the breast pump. The bottom section is downwardly facing when the milk container is positioned such that it is configured to receive milk from the user of the breast pump. In the bottom loaded configuration, when in use the milk flows next to or below the nipple tunnel. That is, the milk container sits substantially below the nipple tunnel when in use.

The bottom loaded configuration may be applied to a milk container with either of an inward or outward configuration (i.e. an inward or outward facing wet section of the milk container).

FIG. 6A shows a bottom loaded configuration milk container with an inward facing wet section (inward configuration). When the milk container 604 is positioned such that it is configured to receive milk from the user of the breast pump, the dry section 632 is located on the side away from the user and the wet section 630 is located on the side towards the user.

FIG. 6B shows a bottom loaded configuration milk container with an outward facing wet section (outward configuration). When the milk container 604 is positioned such that it is configured to receive milk from the user of the breast pump, the wet section 630 is located on the side away from the user and the dry section 632 is located on the side towards the user.

Similar to previously described, the bottom loaded configuration milk containers 604A, 604B shown in FIGS. 6A and 6B may each comprise: a flexible diaphragm 611A, 611B, a first opening 625A, 625B, a second opening 626A, 626B, and an attachment portion 618A, 618B. The flexible diaphragm 611A, 611B may attach to the milk container in a similar manner to as described herein, straightforwardly adapted to the shape of the bottom loaded configuration milk container.

The external walls of the bottom loaded configuration milk containers 604A, 604B comprise a first domed section 622A, 622B and a second domed section 623A, 623B. The first domed section 622A, 622B may be flattened at the bottom to allow the milk container to stand on a surface when not in use. In some embodiments, a bottom loaded milk container comprises a domed section and a substantially flat section.

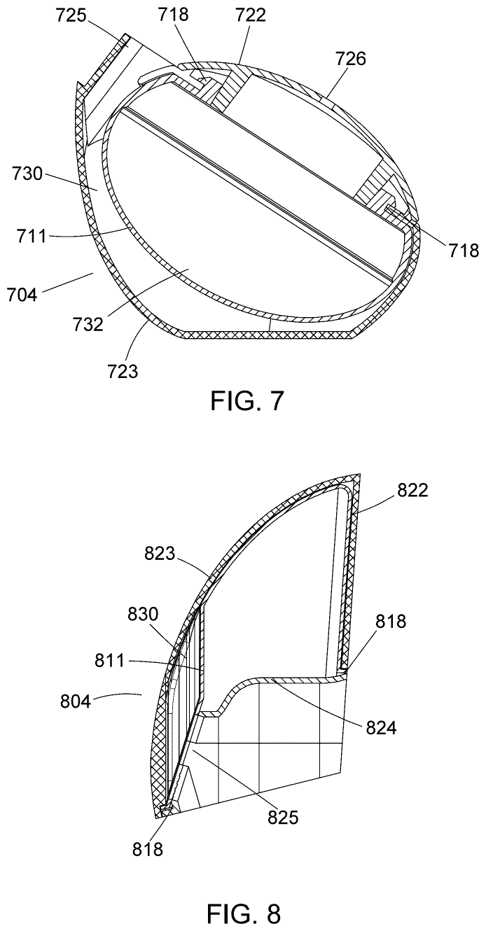

FIG. 7 shows another embodiment of a bottom loaded configuration milk container 704 with an outward facing wet section 730 (outward configuration). In this embodiment, the milk is stored in a similar way as described in relation to other outward configuration milk containers. The following features act in a similar way to as previously described in relation to FIG. 6B: the first opening 725, the dry section 732 and the wet section 730.

The shape of the flexible diaphragm 711, the first domed section 722, and the second domed section are different to the example of FIG. 6B. The first domed section 722 may be a domed bottle cap. The first domed section 722 is smaller than the second domed section 723, the second domed section making up more of the external surface of the milk container. Where the first domed section 722 and second domed section 723 meet there is the attachment portion 718 which attaches the flexible diaphragm to the milk container 704, forming the seal between the wet and dry sections. The flexible diaphragm 711 follows the shape of the second domed section 723. Given the geometry of the first and second domed section, the flexible diaphragm is close to being spherical or ellipsoid shape but with a spherical or ellipsoid hole where the first domed section is.

The second opening 726 may be located along the first domed section 722, the domed bottle cap. When the milk container is not in use, the user may remove the domed bottle cap (the first domed section) and with it the flexible diaphragm. This is so that the user can remove the seal and pour out the collected milk from the wet section 730. In some embodiments, the user may be able to separate the flexible diaphragm from the domed bottle cap. In other examples, the bottle cap and diaphragm are moulded together into one piece.

FIG. 8 shows another embodiment, a top loaded configuration milk container 804. The milk container 804 is positioned such that when it is configured to receive milk from the user of the breast pump, the milk container spans at least a portion of a top section of the breast pump. The top section is upwardly facing when the milk container 804 is positioned such that it is configured to receive milk from the user of the breast pump. The top loaded configuration acts in a similar way to the bottom loaded configuration in that the milk flows from the nipple tunnel through a non-return valve into the wet section 830. As the milk collects, the flexible diaphragm is deformed, the wet section increases in volume whilst the dry section loses volume.

The top loaded configuration milk container is of a segment sphere or ellipsoid shape, in other words a half hemispherical or quarter-ellipsoid shape.

In the top loaded configuration and when the milk container is in use, milk flows next to or above the nipple tunnel. In the bottom loaded configuration, milk flows next to or below the nipple tunnel. Similar to the bottom loaded configuration, the top loaded configuration may be applied to a milk container with either of an inward or outward configuration (i.e. an inward or outward facing wet section of the milk container).

Similar to previously described, the top loaded configuration milk container may comprise: a flexible diaphragm 811, a first opening 825, and a second opening 826.

The milk container 804 may comprise a substantially flat section 822 and a domed section 823. The substantially flat section 822 may comprise an internal protrusion 824 located at the bottom of the substantially flat section. The internal protrusion 824 is away from the edge where the substantially flat section 822 meets the domed section 823. The internal protrusion 824 protrudes within the volume of the milk container, creating an indented portion of the substantially flat section 222. The internal protrusion 224 may be of different sizes depending on the configuration of the milk container and to ensure that it matches the geometry of the nipple tunnel.

The milk container 804 may comprise an attachment portion 818. The attachment portion 818 may be located at the bottom of the substantially flat section 822, where the internal protrusion 824 is, as depicted in FIG. 8. In other examples, the attachment portion 818 may be between the domed section 823 and substantially flat section 822. The flexible diaphragm may attach to the milk container in a similar manner to as described herein, straightforwardly adapted to the shape of the top loaded configuration milk container.

FIG. 9 depicts an example milk container 904 according to some embodiments. In the example of FIG. 9, the flexible diaphragm 911 is configured as a removable milk pouch 940. The milk pouch 940 can be a reusable flexible pouch or milk bag either of which can be used to store the milk without the need to decant the breast milk. When the flexible diaphragm 911 is configured as a removable milk bag 940 it inflates as it is filled with breast milk from the user of the breast pump.

In some embodiments, the milk container 904 may be formed of a first housing piece and a second housing piece, wherein the first housing piece and second housing piece meet at an edge. The first housing piece forms the domed section 923 of the milk container 904 and the second housing piece forms the substantially flat section 922 of the milk container. The first and second housing pieces combine to make a rigid milk container housing 944 as shown in FIG. 9. The removable milk pouch 940 may sit inside the rigid milk container housing 944. When the removable milk pouch 940 inflates as it is filled with breast milk, it may expand up to the volume allowed by the rigid milk container housing 944.

In some embodiments, the milk container 904 may also comprise an attachment plate 942, the attachment plate 942 interfaces the flexible diaphragm 912 with the breast pump (not shown in FIG. 9). The example removable milk bag shown in FIG. 9 is designed for a “top moulded” configuration of milk container (for example, the milk container of FIG. 8). The removable milk pouch 940 of FIG. 9 is a segment sphere shape to align with the segment sphere configuration of a milk container such as in FIG. 8. Of course, a removable milk pouch may also be used for any other milk container configuration disclosed herein. For example, a removable milk bag may be of a breast shaped hemispherical shape to fit within the first and second milk container housing pieces shown in FIG. 2.

The milk container described thus far may comprise a flexible diaphragm located inside the milk container. In some embodiments, the flexible diaphragm forms part of a wall of the milk container. The flexible diaphragm forming part of a wall of the milk container will now be described.

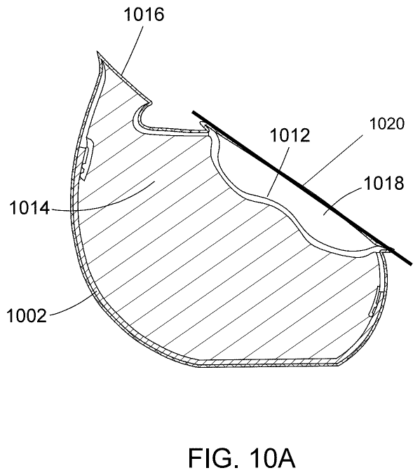

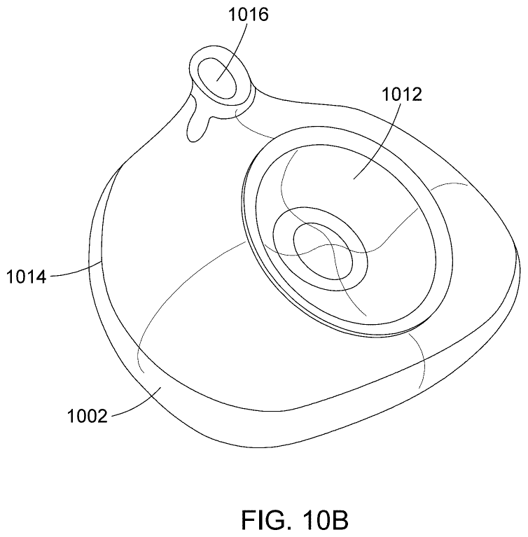

Reference will now be made to FIGS. 10A and 10B. FIG. 10A is a cross sectional view of a milk container 1002. FIG. 10B is a three dimensional view of the milk container 1002.

FIGS. 10A and 10B depict a milk container 1002 according to the present disclosure, wherein the flexible diaphragm forms part of a wall of the milk container. That is, the diaphragm forms part of an external wall of the milk container. In this embodiment, the “dry side” is external to the milk container and the “wet side” is internal to the milk container.

The flexible diaphragm is activated by an air pump applying negative pressure on the dry side of the diaphragm. In some examples, in a relaxed state the diaphragm has a non-flat profile, such as a curved or undulating profile. This enables the diaphragm to move without requiring the diaphragm material to stretch.

A milk container system is comprised of the milk container, the diaphragm forming part of the wall of the container, and an external portion which forms a chamber on the dry side of the diaphragm.

The wet side 1010 of the container is larger than the dry side 1018 of the container system. In FIG. 10A, the wet side 1014 is shown by the dashed area and the dry side 1018 as the non-dashed area. The dry side 1018 is not shown in FIG. 10B. The diaphragm 1012 may have a diameter smaller than the diameter of the milk container 1002. This configuration may be combined with any of the other embodiments described herein.

The diaphragm 1012 may form part of the exterior of the milk container 1002. That is, the diaphragm 1012 may form a wall of the milk container where one side of the diaphragm is internal to the milk container and the other side of the diaphragm is external to the milk container.

The diaphragm 1012 may be sealably attachable to the breast pump housing to form a dry chamber therebetween, defining the “dry side” of the diaphragm. An air pump pumps air into or from the dry chamber to engender movement in the diaphragm to create a suction in the milk container to draw breast milk into the milk container. The wet side and the dry side of the diaphragm in the embodiment in FIGS. 10A and 10B perform the same function as in the other embodiments disclosed herein, and therefore features of those embodiments apply to and can be combined with the embodiment in FIGS. 10A and 10B.

As described above, the breast pump may comprise a housing. A portion of the housing 1020 is illustrated in FIG. 10A. The diaphragm 1012 may be sealably attachable such that it forms a hermetic seal when attached to the housing. The diaphragm 1012 may be attachable such that it can be attached and unattached by the user. The dry chamber 1018 may be formed by the milk container and the housing. FIG. 10A shows the milk container 1002 attached to part of the housing 1020 such that the dry chamber 1018 is formed on the dry side of the diaphragm. FIG. 10B shows the milk container 1002 unattached to the housing such that no dry chamber is formed.

The milk container 1002 may meet the housing at a sealing lip, the sealing lip running circumferentially around the diaphragm. In some examples, the diaphragm comprises the sealing lip. In some examples, the milk container comprises the sealing lip on the wall which meets the diaphragm. The sealing lip may be configured to seal a flat surface 1020 of the housing to form the dry chamber 1018. In some examples, the housing of the breast pump has a flat surface 1020 for sealing to the milk container, such that one wall of the dry chamber is flat. In some examples, the housing has a curved section and a flat surface, where the flat surface abuts the sealing lip to form a seal. This creates a curved wall of the dry chamber 1018, which provide more room for the diaphragm to move into.

The diaphragm 1012 may be overmolded on the milk container to form the wet section 1014 inside the milk container, and the diaphragm sealably attached to the housing to form the dry chamber 1018.

As described above, the milk container 1002 may comprise the diaphragm 1012. The milk container may be removably attachable to the rest of the breast pump by a user. The diaphragm forms part of the milk container and is removably attachable from the breast pump with the other components of the milk container 1002, such that the user can empty milk from the container after a pumping session. The diaphragm 1012 may form part of a wall of the milk container 1002. In other words, the diaphragm 1012 may form part of the milk container surface. For example, the diaphragm 1012 may form around 50%, 30% or 10% of the milk container surface. Any suitable percentage of the milk container surface may be used.

The dry chamber 1018 may comprise a portion of the housing of the breast pump. That is the dry chamber 1018 may be part of the breast pump contained in the same housing as other components of the breast pump, for example, the air pump.

The milk container 1002 may comprise a first opening 1016 for receiving milk from the breast pump along a milk path. The first opening 1016 leads to the wet side. The milk path is similar to the milk path already described, for example the milk flows along the milk path through the breast shield and the nipple tunnel. The dry chamber 1018 may comprise a second opening for connection to an air pump. The second opening may be within the housing.

The first opening 1016 may be for pouring collected milk out of the milk container 1002. Alternatively, the milk container 1002 comprises a separate pouring opening for pouring collected milk out of the milk container.

The diaphragm forms part of the wall of the container. That is, the milk container, into which the milk flows from the milk path, is defined by a body forming walls around an internal chamber for collecting milk. The milk container is connectable to and removable from a milk path having a valve. Milk flows through a valve and into the milk container. The diaphragm forms a wall of the container and therefore is part of the body which is connectable to and removable from the milk path. In use, milk enters the milk container along a milk path, passing a valve before entering the milk container. The milk then collects in the milk container for the duration of the pumping session. The diaphragm forms part of the wall of the milk container. As milk collects in the container it touches the walls of the container. It may therefore fill up against the diaphragm.

The milk comprises an opening for receiving milk along a milk path and a main body for collecting milk. Milk is collected and stored in the main body during the pumping session. The diaphragm forms part of a wall of the main body of the milk container.

The diaphragm 1012 may be located on a portion of the milk container 1002 which, when the breast pump is positioned for use to receive milk from the user of the breast pump, forms a side or an upper portion of the milk container. FIG. 10A shows the breast pump positioned for use to receive milk from the user of the breast pump, this position can be considered upright. As shown in FIG. 10A, the diaphragm 1012 forms an upper portion of the milk container 1002.

In other embodiments, when the breast pump is positioned for use to receive milk from the user of the breast pump, the diaphragm forms a lower portion of the milk container. In these examples, the diaphragm may be configured to have enough spring force to overcome the pressure caused by the weight of the milk pressing down on the diaphragm.

The diaphragm 1012 may be located on the side of the milk container 1002 towards the user, when in its upright position. That is, the side of the milk container which is closer to the user's breast.

The milk container 1002 may have a flat bottom to allow the milk container to be stood on a surface. The diaphragm 1012 may form a side wall of the milk container, and may be on an upper portion of the milk container. That is, the diaphragm may be on a side wall on the upper half of the milk container. Alternatively, the diaphragm may form a top part of the milk container. In other embodiments, the diaphragm may be positioned elsewhere on the milk container.

The milk container 1002 may be a breast shaped hemispherical or half-ellipsoid shape, comprising a domed section and a flexible section. The domed section may be shaped to match or conform with the shape of a breast and/or bra. The shape of the milk container may be described as egg shaped or pebble shaped. The flexible section is the diaphragm 1012. The milk container 1002 may also comprise a substantially flat section at the bottom of the milk container such that the milk container can rest on a surface. In use, when worn by a user, the domed section may face outwards away from the user to conform with a bra. The bottom may be substantially flat, and the diaphragm may be positioned towards the user. The wall portion facing the user during use may be angled (i.e. not vertical).

In other examples, the diaphragm is located on other portions of the milk container. The diaphragm may be positioned in any conceivable location forming a portion of the wall of the main body of the milk container.

The milk container 1002 defines a main internal chamber, and the diaphragm 1012 forms part of the walls of the main internal chamber. That is, the diaphragm forms part of a wall of the main body of the milk container.

The milk container 1002 may comprise a rigid body and a hole, the hole sealed by the diaphragm 1012. Therefore, the walls of the milk container 1002 may comprise a rigid portion (the rigid body) and a flexible portion (the diaphragm 1012). The hole may be substantially the same shape and size as the diaphragm 1012. The hole may be hermetically sealed by the diaphragm 1012.

The diaphragm may be overmolded on the rigid portion of the container, or other forms of attaching the diaphragm to the rigid portions of the container may be used. The diaphragm 1012 may be substantially round, circle and/or oval shaped. The diaphragm 1012 may be a hemispherical bowl shape, a spherical dome and/or a spherical dome shape.

The diaphragm 1012 may be configured to be activated by low air pressure. The diaphragm 1012 may deform under low air pressure, transferring pressure from one side of the diaphragm to the other side. The diaphragm 1012 may oscillate it's position with air pump cycles as the pressure cycles from a higher pressure to a lower pressure.

FIG. 10A shows the milk container 1002 empty of milk. The wet side 1014 forms the entire inside of the milk container 1002. When the milk container 1002 is empty of milk, the wet side 1014 makes up the majority of the total volume of the milk container system while the dry chamber 1018 makes up a smaller volume of the milk container system. Due to the size of the wet side 1014 and the fact it makes up the entire milk container, there is a large amount of air in the wet side when the milk container 1002 is empty of milk.

When milk fills the milk container, the size of the wet side 1014 may increase due to an increased combined volume of air and milk. In turn, the dry side (aka the dry chamber 1018) may decrease in size. This happens by the diaphragm moving outwards from the wet side and into the dry side, hence decreasing the volume of the dry chamber and increasing the volume of the wet side.

Given there is air in the container (i.e. in the wet side) at the beginning of the pumping process, this air takes up internal volume which could otherwise be filled with milk. In order to reach maximum milk capacity of the milk container 1002, so that the container holds much milk as possible, air needs to be vented out of the wet side 1014 in order to make space for the milk. When at maximum capacity of milk, the total volume of the milk container 1002 will be substantially full of milk.

How air may be vented out of the milk container will now be described.

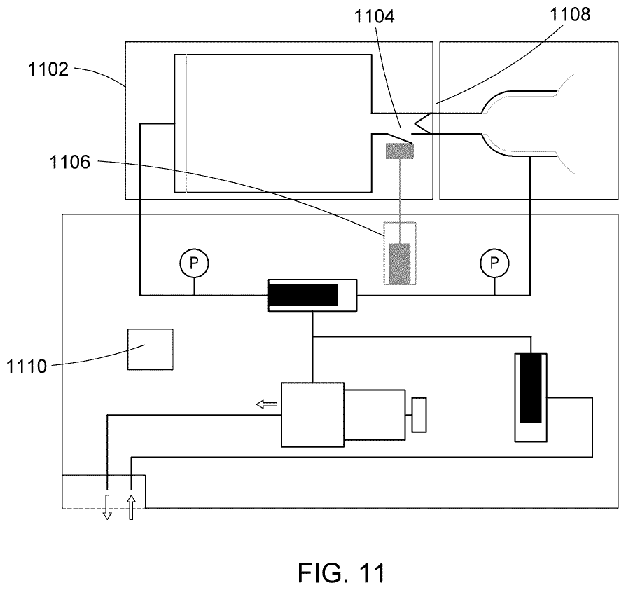

Reference will now be made to FIG. 11.

FIG. 11 depicts a milk container 1102 according to some embodiments. The milk container 1102 may comprise means for venting air. The means for venting air may comprise a valve 1104 located on the wet side of the milk container and an actuator 1106 configured to open the valve.

The valve 1104 may be of any suitable type. One example valve is depicted in FIGS. 5A and 5B. The valve 1104 is located such that when the valve is open the wet side of the milk container is open to the atmosphere. In some embodiments, the valve 404 is located near the non-return valve 1108 which is near the entrance to the milk container. In some other embodiments, the valve 1104 is located in the body of the milk container 1102.

In some embodiments, the milk container 1102 may comprise means for detecting the orientation 1110 of the milk container. The means for detecting the orientation 1110 of the milk container may be any suitable means. For example, an accelerometer, electronic gyroscope and/or an IR system. The IR system may comprise an IR transmitter and receiver, both located inside the milk container. At certain orientations, the milk will block or interfere with the transmitted IR. The IR system is configured to detect a change in orientation.

The means for detecting the orientation 1110 of the milk container may be configured to determine if the milk container is within a predefined range of allowed orientations. The milk container may be configured to vent the air only when the milk container is in the allowed orientations. For example, the allowed orientations may be where the user is upright or near upright. If the user is outside the predefined range of allowed orientations, there is a risk of milk exiting the milk container via the means for venting air.

The means for detecting the orientation as described above may also be present in the embodiments of FIGS. 6, 11 and 9.

In some embodiments, the actuator 1106 may be configured to open the valve 1104 to control the process of venting. The actuator 1106 opens and closes the valve. Excess air held in the milk container is removed due to the diaphragm resetting to the level of the liquid in the bottle. When the dry side of the milk container is equalised to the atmosphere, the diaphragm provides a small positive pressure against the wet side. When the actuator 1106 opens the valve 1104, the small positive pressure is allowed to release. This resets the diaphragm and removes excess air until the diaphragm is restricted by the liquid in the bottle. The diaphragm does not have enough spring force to displace the liquid, only air. That is, the diaphragm has a spring force low enough such that it will not displace liquid but high enough that it will displace excess air.

The actuator 1106 may be a solenoid actuator. Of course, any suitable actuator may be used. When the actuator 1106 closes the valve 1104, no air or indeed liquid can flow out of the valve. When the valve 1104 is closed the pressure in the milk container stays constant. The valve 1104 may be described as an active valve.

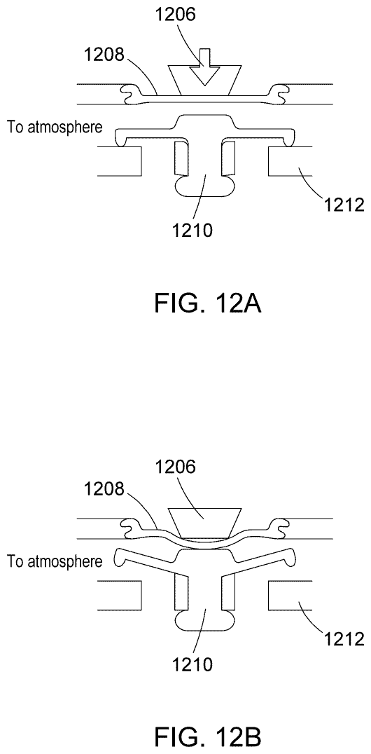

Reference will now be made to FIGS. 12A and 12B.

According to some embodiments, means for venting air comprising a valve located on the wet side of the container and an actuator configured to open the valve is depicted in FIGS. 12A and 12B.

The means for venting air may comprise: an actuator 1206, a flexible sealing portion 1208 and a valve portion 1210. FIG. 12A depicts the actuator 1206 at rest and the valve portion 1210 in the closed position. In the closed position, air is not being vented from the milk container. When the valve portion 1210 is closed, air cannot escape from the milk container. At rest, the actuator 1206 may be in contact with or in close proximity to the flexible sealing portion 1208.

FIG. 12B depicts the actuator 1206 in an engaged position which causes the valve portion 1210 to be in the open position. In the open position, air is vented from the milk container. When the actuator 1206 is engaged, the actuator 1206 makes contact with the flexible sealing portion 1208, flexing the flexible sealing portion 1208 and flexing the valve portion 1210. When flexed, the valve portion 1210 is open, allowing air to escape from the milk container. In the open position, air is vented from the milk container via an air channel between the flexible sealing portion 1208 and the milk container wall 1212. The air channel connects to the atmosphere external to the breast pump system.

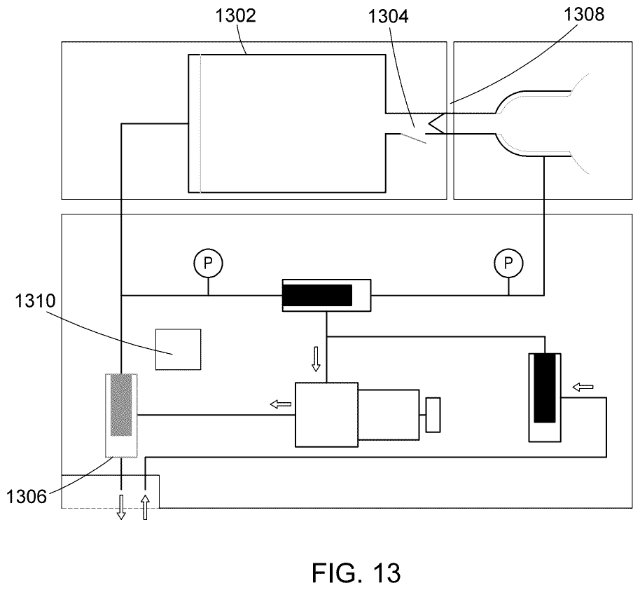

Reference will now be made to FIG. 13.

FIG. 13 depicts a milk container 1302 according to some embodiments. The milk container 1302 may comprise means for venting air. The means for venting air may comprise a valve 1304 located on the wet side of the container configured to open in response to air applied to the dry side of the container.

The breast pump system may also comprise a means for pressuring the milk container 1306. For example, the means for pressuring the milk container 1306 may be a solenoid. The means for pressuring the milk container 1306 may be a pump connected to the dry side of the milk container. The means for pressuring the milk container 1306 may be configured to increase the pressure on the dry side of the milk container. This causes the dry side to increase in volume, causing the wet side to decrease in volume and vent out air from the milk container and towards the non-return valve 1308. The vented air can escape through the valve 1304, such as a non-return valve, flap valve, membrane microvalve, ball microvalve, etc. The valve 1304 is a one-way valve which is configured to only allow vented air from the milk container to escape. The valve 1304 does not let atmospheric air enter the system.

In some embodiments, the milk container 1302 may comprise means for detecting the orientation 1313 of the milk container. The means for detecting the orientation 1313 of the milk container may operate in a similar way as described in relation to FIG. 11.

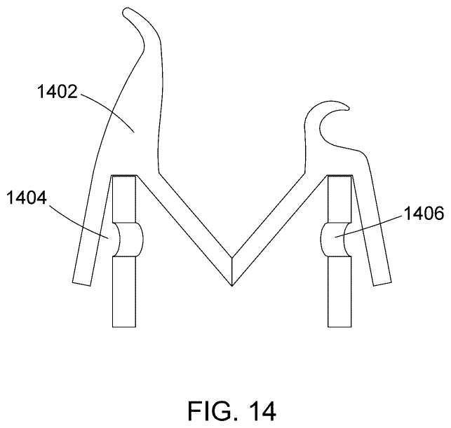

Reference will now be made to FIG. 14.

In some examples, the valve 1004 located on the wet side of the container configured to open in response to air applied to the dry side of the container may be integrated into the non-return valve 1008. An example is depicted in FIG. 14. There is a first valve 1404 on one side of the wall of the nipple tunnel and a second valve 1406 on the opposite side of the nipple tunnel. The nipple tunnel may be defined as a channel between the milk container and the breast shield.

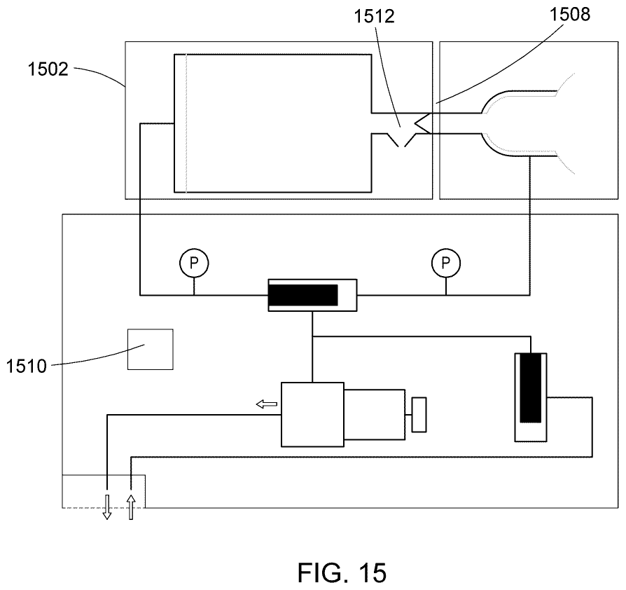

Reference will now be made to FIG. 15.

FIG. 15 depicts a milk container 1502 according to some embodiments, wherein the means for venting air comprises a non-return valve 1512 located on the wet side of the container. The non-return valve 1512 may be described as the second non-return valve, an additional non-return valve to the first non-return valve 1508 which separates the milk container and breast shield. The non-return valve 1512 may be located on any suitable part of the wet side of the container.

The breast pump may be configured to pump the milk container 1502 to atmospheric pressure, such that excess air in the wet side is expelled via the non-return valve 1512. This configuration depicted in FIG. 15 is advantageous because it makes use of the breast pump's existing architecture without the need for additional components such as actuators or solenoids.

The non-return valve 1512 remains closed during a pumping phase of the operation of the breast pump. The non-return valve 1512 opens to expel excess air in the air venting phase of operation.

In some embodiments, the milk container 1502 may comprise means for detecting the orientation 1510 of the milk container. The means for detecting the orientation 1510 of the milk container may operate in a similar way as described in relation to FIG. 4.

As described above, a milk-volume measurement process may measure the volume of milk and/or air in the milk container by changing the pressure or volume of the milk container. In one embodiment, the milk container 1502 may be configured to vent air from the non-return valve 1512 during the milk-volume measurement process. As the pressure or volume of the milk container changes, it may push out excess air which is expelled through the non-return valve 1512. This may occur every time the milk-volume measurement process is run.

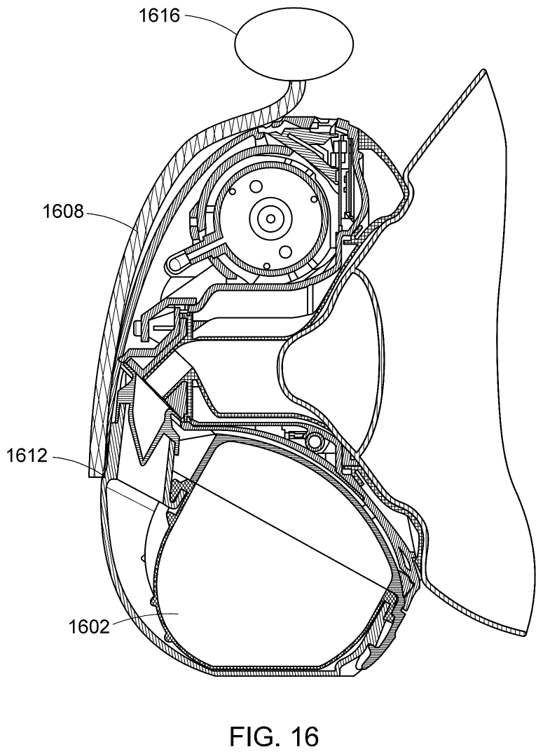

Reference will now be made to FIG. 16.

FIG. 16 depicts a milk container 1602 according to some embodiments, wherein the means for venting air comprises an opening 1612 on the wet side, a tube 1608 connected to the opening, and a valve 1616 connected to the tube and located on an external side of the breast pump, wherein the valve is configured to be actuated by a user.

The valve 1616 may be configured to be user accessible. For example, the valve 1616 may be located on the topmost point of the breast pump milk container system for easy access.

This configuration allows the user to relieve air pressure/excess air whilst pumping. Advantageously, this configuration can also allow for a quick decant of milk for high volume milk producer users. To decant, the user would need to actuate the valve 1616 and move the orientation of the system such that the milk flows from the milk container 1602 and out of the valve. The valve 1616 may be any suitable valve.

The non-return valves provided herein may have a small cracking pressure to overcome. This makes the milk container resilient to leaks even when there is no vacuum inside the container. This is useful, for example, when the pump is off but the bottle is full of milk and the vent valve is covered by milk. If there were no cracking pressure, just the weight of the milk would be enough for it to open and milk could leak out. If there is a cracking pressure, the valve will remain shut unless the pressure inside the bottle is greater than the cracking pressure.

Throughout the description it is described that air is vented out. However, more broadly, it is possible to vent out any positive pressure according to the present disclosure.

The milk container is configured to provide one or more of the following advantageous effects:
A more efficient pumping system due to minimal air in the system and the base level vacuum.
No milk leakage during use, the milk container allows a base level vacuum to be generated and the milk container comprises non-return valves. The milk container of the present disclosure also overcomes the need for an opening to the external environment or a “breather hole”. An opening from the milk container to the external environment can cause leaks. The configuration of the diaphragm inside the milk container or use of the milk pouch overcomes the need for a breather hole.
The milk container also allows for pumping in any orientation, as a result of not having a breather hole and having the flexible diaphragm provide backflow protection to the pump.
Compact in-bra wearable device, either fully in-bra or in-bra milk collection with separate pump
Easy to clean and dry, by ensuring only certain aspects of the milk container are in contact with milk production.
Discrete, both the shape, volume and ability to check milk flow easily
Improved comfort, a base level vacuum has been seen to improve comfort, this is likely due to the decrease in rubbing between the breast shield and nipple as the breast shield is tightly fitted to the breast

Features of the above embodiments can be combined in any suitable manner. It will be understood that the above description is of specific embodiments by way of aspect only and that many modifications and alterations will be within the skilled person's reach and are intended to be covered by the scope of the appendant claims.

The disclosure also includes the following clauses.
1. A milk container for a breast pump, comprising a flexible diaphragm located inside the milk container.
2. The milk container of clause 1 being configured to be attached to a milk pumping system to receive milk from a user of the breast pump.
3. The milk container of any preceding clause, wherein the milk container is a re-useable milk container with a curved or breast-shaped surface.
4. The milk container of clause 2, wherein the flexible diaphragm is a milk barrier for preventing milk from exiting the milk container and for preventing milk from entering the milk pumping system.
5. The milk container of any preceding clause, wherein the flexible diaphragm is designed to deform when acted upon by a vacuum and/or wherein the flexible diaphragm is made, at least in part, from silicone.
6. The milk container of any preceding clause, wherein the flexible diaphragm forms a hermetic seal around an internal wall of the milk container.
7. The milk container of any preceding clause configured to be attached to a breast pumping chamber, the milk container comprising a first opening for receiving milk from the breast pump along a milk path, and a second opening for connection to an air pump.
8. The milk container of clause 7, wherein the flexible diaphragm is located between the first and second opening and forms a seal.
9. The milk container of clause 7, wherein the flexible diaphragm is configured to deform under a negative pressure from the air pump and create a suction along the milk path.
10. The milk container of any of clauses 7 to 9, wherein the first opening is for pouring collected milk out of the milk container.
11. The milk container of any preceding clause, wherein the milk container comprises a wet section for receiving breast milk and a dry section for holding air and the flexible diaphragm separates the wet section and the dry section.
12. The milk container of any preceding clause, wherein:
   the milk container is a breast shaped hemispherical or half-ellipsoid shape, comprising a substantially flat section and a domed section, wherein the flat section and domed section meet at an edge; or
   wherein the milk container is a spherical or ellipsoid shape, comprising a first domed section and a second domed section.
13. The milk container of any preceding clause being configured to be in-bra wearable.
14. The milk container of any preceding clause, wherein, in use when the milk container is positioned such that it is configured to receive milk from the user of the breast pump, the wet section is located on the side away from the user and the dry section is located on the side towards the user and/or wherein the wet section is configured to be visible to the user.
15. The milk container of clause 14, wherein the wet section forms a ring-shaped chamber.
16. The milk container of any preceding clause, wherein the flexible diaphragm comprises:
   a rigid outer ring,
   a flexible diaphragm portion, and
   a rigid central core.
17. The milk container of clause 16, wherein the rigid central core comprises a non-return valve.
18. The milk container of any of clauses 16 to 17, wherein the milk container comprises a domed section and wherein the rigid outer ring:
   extends circumferentially around the domed section;
   is located at the edge of the milk container; and/or
   is located along an axis extending from substantially the centre of the domed section.
19. The milk container of any of clauses 16 to 18, wherein the flexible diaphragm portion connects the rigid outer ring and the rigid central core.
20. The milk container of any of clauses 16 to 19, wherein a milk channel passes through the rigid central core to deliver milk from a user's breast into the wet section.
21. The milk container of any of clauses 1 to 13, wherein, when the milk container is positioned such that it is configured to receive milk from the user of the breast pump the dry section is located on the side away from the user and the wet section is located on the side towards the user.
22. The milk container of any of clauses 1 to 13 or 21, wherein the milk container is formed of a first housing piece and a second housing piece, wherein the first housing piece and second housing piece meet at an edge.
23. The milk container of clause 22, wherein:
   the first housing piece is shaped to be substantially flat and the second housing piece is shaped as a breast shaped hemispherical or ellipsoid dome; or
   the first housing piece is shaped as a hemispherical or ellipsoid dome and the second housing piece is shaped as a hemispherical or ellipsoid dome.
24. The milk container of clause 23, wherein the first housing piece comprises an attachment portion to receive a portion of the flexible diaphragm and seal the flexible diaphragm to the first housing piece.
25. The milk container of clause 24, wherein the attachment portion is located along at least a portion of the edge and hermetically seals the flexible diaphragm to the edge.
26. The milk container of any of clauses 22 to 25, wherein the flexible diaphragm is integrally formed and/or is transparent or optically clear.
27. The milk container of any of clauses 22 to 26, wherein the milk container comprises a domed section and a first opening configured to receive milk from a user's breast located along an axis extending from substantially the center of the domed section.
28. The milk container of any of clauses 22 to 27, wherein the second opening configured to provide an air path to an air pump for generating a vacuum is located at the edge of the first housing piece and second housing piece.
29. The milk container of any preceding clause, wherein, when the milk container is positioned such that it is configured to receive milk from the user of the breast pump, the milk container spans one of:
   at least a portion of a front section of the breast pump, wherein the front section is outwardly facing away from the user;
   at least a portion of a top section of the breast pump, wherein the top section is upwardly facing;
   at least a portion of a bottom section of the breast pump, wherein the bottom section is downwardly facing.
30. The milk container of any of clauses 14 to 15, wherein when the flexible diaphragm is attached to the milk container via a bottle cap, wherein the bottle cap removes the flexible diaphragm from the milk container.
31. The milk container of any preceding clause, wherein the flexible diaphragm is configured as a removable flexible pouch or milk bag that inflates as it is filled with breast milk from the user of the breast pump.
32. The milk container of any preceding clause, wherein:
   the flexible diaphragm has a spring force which is less than the weight of the volume of milk that can fit in the container, in any position of the diaphragm; or
   the flexible diaphragm has a spring force greater than the weight of the volume of milk the can fit in the container, in any position of the diaphragm.
33. A breast pump comprising the milk container of any preceding clause.
34. The breast pump of clause 33, configured as a self-contained, in-bra wearable device.
35. The breast pump of any of clauses 33 to 34, wherein the milk container comprises a second opening configured to provide an air path to an air pump for generating a vacuum and wherein the breast pump comprises a first pressure sensor between the air pump and the milk container and a second pressure sensor between the air pump and a breast shield.
36. A method for filling a milk container with breast milk from a breast pump,
   providing a flexible diaphragm inside the milk container to hermetically seal a dry section from a wet section;
   inserting milk from a first opening into the wet section, thereby causing the area of the dry section to decrease and the area of the wet section to increase.
37. The method of clause 36, further comprising drawing a vacuum in the dry section.
B1. A wearable breast pump comprising:
   a milk pumping system for receiving milk from a user of the breast pump; and
   a milk container comprising a flexible diaphragm,
   wherein the flexible diaphragm forms part of a wall of the milk container.
B2. The breast pump of clause B1, wherein the flexible diaphragm forms part of the exterior of the milk container.
B3. The breast pump of clause B1 or clause B2, wherein the flexible diaphragm is smaller than the diameter of the milk container.
B4. The breast pump of any of clauses B1 to B3, wherein the breast pump comprises a housing, and wherein the flexible diaphragm is sealably attachable to the housing to form a dry chamber therebetween.
B5. The breast pump of clause B4, wherein the milk container comprises a first opening for receiving milk from the breast pump along a milk path, and wherein the dry chamber comprises a second opening for connection to an air pump.
B6. The breast pump of clause B5, wherein the first opening is for pouring collected milk out of the milk container, or wherein the milk container comprises a separate pouring opening for pouring collected milk out of the milk container.
B7. The breast pump of any of clauses B1 to B6, wherein the diaphragm is located on a portion of the milk container which, when the breast pump is positioned for use to receive milk from the user of the breast pump, forms a side or an upper portion of the milk container.
B8. The breast pump of any of clauses B1 to B7, wherein the diaphragm forms part of the wall of the milk container wherein, when the breast pump is positioned for use to receive milk from the user of the breast pump, the diaphragm is located on the side towards the user.
B9. The breast pump of any of clauses B1 to B8, wherein the flexible diaphragm is a milk barrier for preventing milk from exiting the milk container and for preventing milk from entering the milk pumping system.
B10. The breast pump of any of clauses B1 to B9, wherein the flexible diaphragm is designed to deform when acted upon by a vacuum and/or wherein the flexible diaphragm is made, at least in part, from silicone, a rubber or an elastomer.
B11. The breast pump of any of clauses B1 to B10, wherein milk is received into the milk container along a milk path and the milk container is connectable to and removable from the milk path.
B12. The breast pump of any of clauses B1 to B11, wherein the milk container is removably attachable to the rest of the breast pump by a user.
B13. The breast pump of any of clauses B1 to B12, wherein the diaphragm forms 50%, 30% or 10% of the milk container surface.
B14. The breast pump of any of clauses B1 to B13, wherein the milk container comprises a rigid body and a hole, the hole sealed by the diaphragm.
B15. The breast pump of any of clauses B1 to B14, wherein the diaphragm is round or oval shaped.
B16. The breast pump of any of clauses B1 to B15, wherein the diaphragm is configured to be activated by low air pressure.
B17. The breast pump of any of clauses B1 to B16, wherein the milk container comprises a wet section for receiving breast milk, wherein the diaphragm separates the wet section and a dry chamber for holding air.
B18. The breast pump of clause B17, wherein the wet side is larger than the dry chamber.
B19. The breast pump of clause B17 or clause B18, wherein the diaphragm is overmolded on the milk container to form the wet section inside the milk container, and the diaphragm is sealably attached to the housing to form the dry chamber.
B20. A milk container for use with the breast pump of any of clauses B1 to B19.

1. A wearable breast pump comprising:
a milk pumping system to receive milk from a user of the breast pump; and
a milk container comprising a flexible diaphragm,
wherein the flexible diaphragm forms part of a wall of the milk container.

2. A wearable breast pump comprising:
a milk pumping system to receive milk from a user of the breast pump; and
a milk container comprising a flexible diaphragm,
wherein the flexible diaphragm is located inside the milk container.

3. The breast pump of claim 2, wherein the flexible diaphragm intersects the milk container.

4. The breast pump of claim 2, wherein the flexible diaphragm forms a hermetic seal around an internal wall of the milk container.

5. The breast pump of any of claim 2, configured to be attached to a breast pumping chamber, the milk container comprising a first opening for receiving milk from the breast pumping chamber along a milk path, and a second opening for connection to an air pump.

6-7. (canceled)

8. The breast pump of claim 1, wherein the flexible diaphragm forms part of the exterior of the milk container.

9. The breast pump of claim 1, wherein the flexible diaphragm is smaller than the diameter of the milk container.

10. The breast pump of any of claim 1, wherein the breast pump comprises a housing, and wherein the flexible diaphragm is sealably attachable to the housing to form a chamber therebetween.

11-13. (canceled)

14. The breast pump of claim 1, diaphragm forms part of the wall of the milk container wherein, when the breast pump is positioned for use to receive milk from the user of the breast pump, the diaphragm is located on the side towards the user.

15. The breast pump of claim 1, wherein the flexible diaphragm is a milk barrier for preventing milk from exiting the milk container and for preventing milk from entering the milk pumping system.

16. (canceled)

17. The breast pump of claim 1, wherein the milk container comprises a wet section for receiving breast milk and a dry section for holding air and the flexible diaphragm separates the wet section and the dry section, and wherein the wet section of the milk container comprises: the flexible diaphragm and an internal wall of the milk container.

18. (canceled)

19. The breast pump of claim 1, wherein:
the milk container is a breast shaped hemispherical or half-ellipsoid shape, comprising a substantially flat section and a domed section, wherein the flat section and domed section meet at an edge; or
wherein the milk container is a spherical or ellipsoid shape, comprising a first domed section and a second domed section.

20. The breast pump of claim 17, wherein, in use when the milk container is positioned such that it is configured to receive milk from the user of the breast pump, the wet section is located on the side away from the user and the dry section is located on the side towards the user, and/or wherein the wet section is configured to be visible to the user.

21. (canceled)

22. The breast pump of claim 1, wherein the flexible diaphragm comprises:
a rigid outer ring,
a flexible diaphragm portion, and
a rigid central core.

23. The breast pump of claim 22, wherein the rigid central core comprises a non-return valve.

24. The breast pump of claim 22, or wherein the milk container comprises a domed section and wherein the rigid outer ring:
extends circumferentially around the domed section;
is located at the edge of the milk container; and/or
rigid central core is located along an axis extending from substantially the centre of the domed section.

25-26. (canceled)

27. The breast pump of claim 17, wherein, when the milk container is positioned such that it is configured to receive milk from the user of the breast pump the dry section is located on the side away from the user and the wet section is located on the side towards the user.

28. The breast pump of claim 1, wherein the milk container is formed of a first housing piece and a second housing piece, wherein the first housing piece and second housing piece meet at an edge.

29-30. (canceled)

31. The breast pump of claim 1, wherein, when the milk container is positioned such that it is configured to receive milk from the user of the breast pump, the milk container spans one of:
at least a portion of a front section of the breast pump, wherein the front section is outwardly facing away from the user;
at least a portion of a top section of the breast pump, wherein the top section is upwardly facing;
at least a portion of a bottom section of the breast pump, wherein the bottom section is downwardly facing.

32. (canceled)

33. The breast pump of claim 1, wherein:
the flexible diaphragm has a spring force which is less than the weight of the volume of milk that can fit in the container, in any position of the diaphragm; or
the flexible diaphragm has a spring force greater than the weight of the volume of milk the can fit in the container, in any position of the diaphragm.

34-36. (canceled)
